(19) United States
(12) Patent Application Publication
(10) Pub. No.: US 2011/0087172 A1
(43) Pub. Date: Apr. 14, 2011

(54) ANAL MEDICATION APPLICATOR EXTENSION

(76) Inventor: Vernon Wesley Gunderson, Las Vegas, NV (US)

(21) Appl. No.: 12/577,308

(22) Filed: Oct. 12, 2009

(51) Int. Cl. A61M 31/00

(52) U.S. Cl. 604/181

(57) ABSTRACT

Disclosed is applicator extension device including a semi rigid elongate tube bent at an angle and an adjustably bendable elongate tube each having a first end fitting configured to be removably attached to the opening of a medicine container and a second end terminating in a second end fitting configured to be removably attached to an applicator. Embodiments of this device include embodiments wherein all or a portion of the tube is made of non-rebound tubing or stay put tubing; wherein a flange is circumferentially fixed on the outer surface of the elongate tube adjacent to the end of the elongate tube adjacent to the applicator; wherein the end fittings are releasably attached to either or both ends of the elongate tube; and/or wherein a wherein a flexible tube is attached to the elongate tube, preferably between the end of the elongate tube and the opening of the medicine container.

### FIELD OF THE DISCLOSURE

This invention relates to an applicator extension for applying ointments, creams and other high-viscosity liquid medications from a medicine container through an applicator to the anal-genital area.

### BACKGROUND

Anal-genital medication, such as ointments, foams, gels and liquids dispensed from tubes or other containers, has been administered both topically to the anal/genital region and intrarectally/intravaginally generally using short, straight applicators supplied with the medicine container. For example, hemorrhoid ointment is commonly dispensed from a tube of ointment by way of a short applicator attached to the opening of the tube of ointment. Specifically, the applicator is attached directly to the tube of ointment and inserted a short way into the rectum or vagina. However, application in this manner can be awkward. For example, some individuals may have physical limitations or disabilities making it difficult to reach the rectum or vagina. Additionally, any person trying to apply the ointment while sitting on the toilet may find it difficult to do so without dipping their hands or the tube of ointment into the water in the toilet bowl.

Prior art devices for applying liquid medications to the anal-genital region leave much to be desired from the standpoint of ease of use, adjustability, and connectivity with standard medicine containers particularly with regard to higher viscosity liquid and semi-solid medications such as ointments and creams. For example, U.S. Pat. Nos. 5,250,024 (Kensey) and 5,380,275 (Kensey and Kaufmann) teach delivery of low viscosity fluids such as enemas, soapy water, salt water or oils from a squeeze bottle or pumping device into the anus or vagina. U.S. Pat. No. 6,632,195 likewise teaches delivery of low viscosity fluids from a squeeze bottle to the anus and other areas of the body by means of an enema tip, douche tip or bidet tip. The '195, '275 and '024 patents simply do not reference any use of a high viscosity fluid such as an ointment or cream. U.S. Pat. No. 5,397,312 teaches delivery of a cream-like substance into the vagina via a device in which the part of the device to be inserted into the vagina is curved to approximate the shape of the vagina but the device cannot be connected to a medicine container and is not adjustable.

The present invention is directed to a simple device that addresses the need for an easy to use, optionally adjustable, device that will convey high viscosity fluids such as hemorrhoid ointments and creams from the original medicine container to the user's anal-genital region. Although the description below uses a tube of ointment as an example, the applicator extension can be used with a wide variety of ointment, foam, gel and liquid medicine containers.

### SUMMARY OF THE INVENTION

Disclosed herein is an applicator extension for applying medication from a medicine container through an applicator to the anal-genital area. The applicator extension includes an elongate tube bent at an angle with a first end terminating in a first end fitting configured to be removably attached to the opening of the medicine container and a second end terminating in a second end fitting configured to be removably attached to the applicator. In one embodiment, the elongate tube is bent at an angle of approximately 90 degrees. In one embodiment configured to fit many standard configurations of medicine containers, e.g., tubes of ointment, with threaded caps and applicator tips, the applicator extension includes a female threaded fitting at one end to screw onto a male threaded fitting of the opening of a medicine container and a second end male threaded end fitting configured to screw into a female threaded fitting of the applicator. In other embodiments, either or both of the end fittings are releasably attached to the elongate tubes so as to allow for a variety of fittings to be attached allowing the applicator extension to work with variously sized and configured medicine containers. Embodiments of the applicator extension could also include a flange circumferentially fixed on the outer surface of the elongate tube adjacent to the second end to limit the length of penetration of the applicator into the user's anal or vaginal canals. In other embodiments the angle at which the elongate tube is bent may be user adjustable. In these embodiments all or a portion of the elongate tube may be made of non-rebound tubing, stay put tubing or similar materials that substantially hold their shape once bent. In another embodiment, all or a portion of the elongate tube may be constructed of an accordion-type pleat material allowing the elongate tube or a portion thereof to be snapped into varying angles by the user. In yet another embodiment the applicator extension could include a flexible tube preferably attached to the end of the elongate tube to be attached to the opening of the medicine container. This embodiment allows the user even more flexibility in using the applicator extension. Like the applicator extension incorporating a substantially rigid elongate tube, the embodiments of the applicator extension incorporating the user adjustable could also have a female threaded end fitting configured to screw onto a male threaded fitting of the opening of the medicine container and a second male threaded end fitting configured to screw into a female threaded fitting of the applicator; a flange circumferentially fixed on the outer surface of the elongate tube adjacent to end of the elongate tube adjacent the applicator to limit the insertion of the applicator into the rectal or vaginal canals; and/or releasably attached end fittings at either or both ends of the elongate tube. Also disclosed is applicator extension device including an adjustably bendable elongate tube having a first end fitting configured to be removably attached to the opening of a medicine container and a second end terminating in a second end fitting configured to be removably attached to an applicator. Embodiments this device include embodiments wherein all or a portion of the tube is made of non-rebound tubing or stay put tubing; wherein a flange is circumferentially fixed on the outer surface of the elongate tube adjacent to the end of the elongate tube adjacent to the applicator; wherein the end fittings are releasably attached to either or both ends of the elongate tube; and/or wherein a wherein a flexible tube is attached to the elongate tube, preferably between the end of the elongate tube and the opening of the medicine container.

### BRIEF DESCRIPTION OF THE DRAWINGS

These and other features and advantages will become appreciated as the same becomes understood with reference to the description, claims and drawings wherein:

FIG. 1 is an exploded perspective view of one embodiment of the device;

FIG. 2 is a perspective view of an embodiment of the device;

FIG. 3 is a perspective view of another embodiment of the device;

FIG. 4 is a perspective view of another embodiment of the device;

FIG. 5 is a perspective view of one embodiment of the device in use for delivering ointment; and

FIG. 6 is a perspective view of one embodiment of the device in use for delivering ointment.

### DESCRIPTION

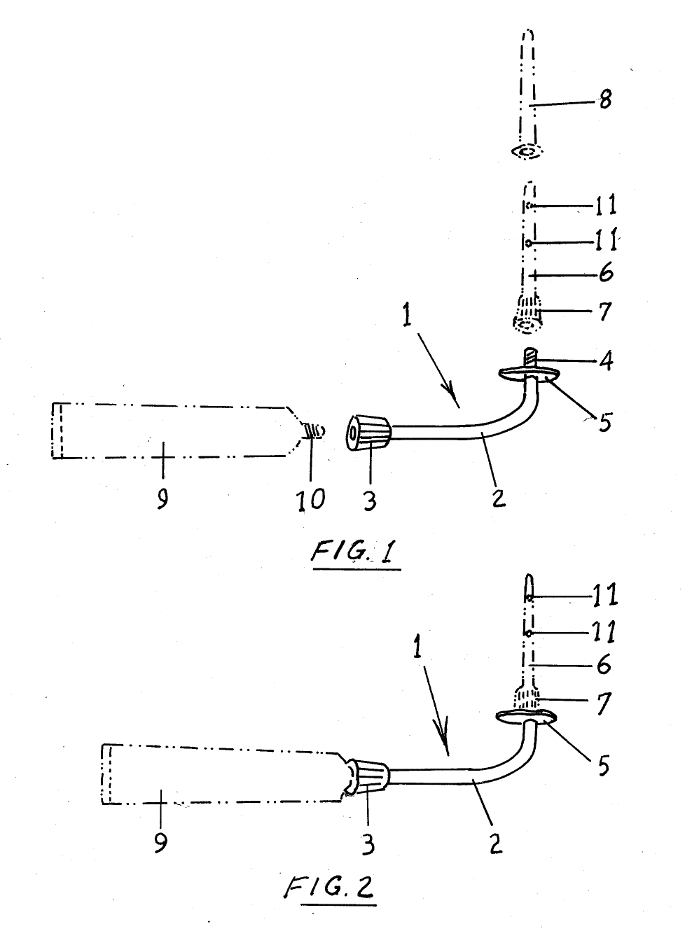

Shown in FIG. 1 and FIG. 2 is a typical tube of ointment 9 a first end of which is sealed and a second end of which has an opening 10 through which ointment is dispensed when the tube of ointment 9 is squeezed. Although the invention is described with reference to ointment being dispensed from a tube, the invention can be used with a wide variety of liquid and semi-solid medications including but not limited to high viscosity medications such as ointments and creams and for the purposes of this description the terms ointment and cream are used synonymously. Also, although other configurations of containers other than tubes are possible the invention is herein described in use with a tube. Thus, the outer circumference of the opening 10 generally has a standard male thread. When purchased, the tube of ointment 9 may have a cap (not shown) configured with female threads on the interior circumference of the cap to engage the male threads on the opening 10 in the tube of ointment 9 so as to contain the ointment in the tube of ointment 9. The tube of ointment 9 may also be supplied with a hollow applicator 6 a first end 7 of which is configured with female threads on the interior circumference to engage the threads on the opening 10 of the tube of ointment 9. The second end of the applicator 6 may simply contain an opening through which ointment is dispensed (not shown) or, as shown, the outer surface of the applicator may contain perforations 11 open to the interior of the applicator 6 through which the ointment is dispensed. The purpose of the perforations 11 is to supply ointment primarily to the circumferential area immediately inside the anus. Also generally supplied with the tube of ointment 9 is an applicator cap 8 configured to cover the applicator 6 and contain ointment in the tube of ointment 9 when not in use.

One embodiment of the applicator extension 1 is shown in FIG. 1 in an exploded view with the apparatus generally supplied with the tube of ointment 9. The same embodiment is shown in FIG. 2 as it would be attached to the tube of ointment 9 and the apparatus generally supplied with the tube of ointment 9. The applicator extension 1 includes a semi-rigid elongate tube 2 bent, in one embodiment, at roughly a 90-degree angle. Although the elongate tube 2 must be sufficiently rigid to hold the angle, it need not be entirely non-pliable or non-flexible. In one embodiment, the elongate tube 2 is made of material identical or similar to that used to make the applicator 6 supplied with the tube of ointment 9. However, the elongate tube 2 could be made of plastic (e.g., polystyrene, PVC, acrylic, polyethylene, etc.), rubber, metal or other suitable material. It would be preferable for the elongate tube 2 to be of a transparent or translucent material to allow the user to be able to see the advancement of the ointment through the elongate tube 2.

With continuing reference to FIG. 1 and FIG. 2, the elongate tube 2 terminates at a first end in a first end fitting 3 configured to be releasably attached to the opening 10 of the tube of ointment 9. The elongate tube 2 terminates at a second end in a second end fitting 4 configured to be releasably attached to the first end of the applicator 7. Most tubes of ointment 9 have a male threaded fitting at the opening 10 and a female threaded fitting on the first end of the applicator 7. When used without the invention, the female threaded fitting 7 of the supplied applicator 6 is threaded onto the male threaded opening 10 of the tube of ointment 9. The applicator extension is designed to be placed between the opening 10 of the tube of ointment 9 and the first end of the applicator 7 that was supplied with the tube of ointment 9. Thus, in one embodiment of the applicator extension 1 configured to be used with a tube of ointment 9 as just described, the first end fitting 3 and the second end fitting 4 of the elongate tube 2 are threaded so as to be able to be screwed on and off of the threaded opening 10 of the tube of ointment 9 and the threaded first end of the applicator 7. Specifically, in one embodiment to be configured with a tube of ointment 9 having a threaded male opening 10, the first end fitting 3 of the elongate tube 2 would be a female threaded fitting and the second end fitting 4 of the elongate tube 2 would be a male threaded fitting.

It is typical that tubes of ointment come in several different sizes. Each size may have differently sized and configured openings 10 and differently sized and configured applicators 6 with differently sized and configured first end fittings 7 of the applicators 6. Moreover, anal-genital medications and the like may come in a wide variety of medicine containers all with differently sized and configured openings and applicators. Accordingly different embodiments of the applicator extension 1 may have different sized and configured first end fittings 3 and second end fittings 4 to accommodate differently sized and configured medicine containers. In alternative embodiments the first end fittings 3 and second end fittings 4 of the applicator extension 1 are releasably attached to the elongate tube 2. In these embodiments, various first end fittings 3 and second end fittings 4 could be releasably attached to the elongate tube 2 as appropriate to the size and type of the opening 10 and applicator first end fitting 7 of variously configured medicine containers 9 that the user 12 may have. In other embodiments, end fitting adaptors could be removably attached to the first end fittings 3 and the second end fittings 4 which are permanently attached to the elongate tube 2 to accomplish the same result. Other embodiments of the applicator extension 1 could be supplied with a custom applicator 6 configured to be attached to the second end fitting 4 of the elongate tube 2. Such would be useful where an applicator 6 is not supplied with the medicine containers 9 or for alternative methods of applying the medicine other than that provided by the applicator 6 supplied with the medicine container 9.

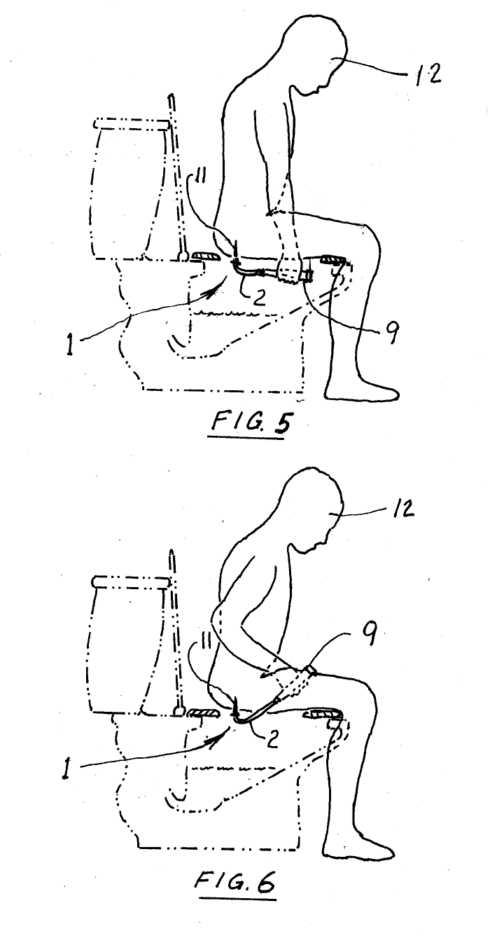

With reference to FIGS. 2, 3, 4, 5 and 6, a typical tube of ointment 9 with the applicator 6 supplied with the tube of ointment 9 requires the user while seated on a toilet to orient the tube of ointment 9 vertically or nearly vertically and to place the user's hand or hands substantially within the toilet bowl. As seen in FIGS. 2, 3, 4, 5, and 6 the applicator extension 1 provides an extended fluid communication between the opening 10 of the tube of ointment 9 and the applicator 6 and changes the angle of application of the ointment. Thus in one embodiment (seen in FIG. 2), the first end fitting 3 of the applicator extension 1 is screwed onto the opening 10 (not viewable in FIG. 2) of a tube of ointment 9. The second end fitting 4 (not viewable in FIG. 2) is screwed into the first end fitting 7 of the applicator 6. When the tube of ointment 9 is squeezed the ointment is forced out of the opening 10 of the tube of ointment 9 and travels through the elongate tube 2 and into the applicator 6 and is discharged from the applicator at some distance from the opening and at an angle other than that when it was discharged from the opening 10. As seen in FIGS. 5 and 6, this allows the user 12 to apply the ointment while holding the ointment tube 9 horizontally (see FIG. 5 for example) or at other angles convenient to the user 12 (see FIG. 6 for example). In the embodiment shown in FIG. 1 and FIG. 2 an optional flange 5 is circumferentially attached to the exterior of the elongate tube 2 adjacent to the second end fitting 4 of the elongate tube 2. The flange 5 prevents the applicator 6 from being inserted too far into the anal canal and ensures that the medicine is applied to the circumferential area immediately inside the anus.

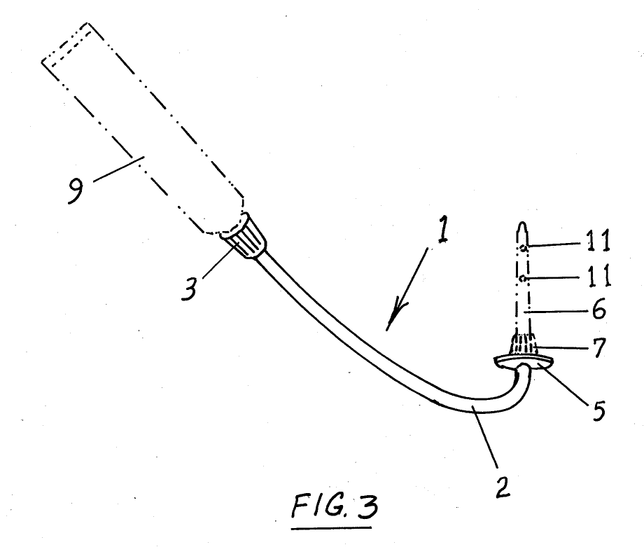

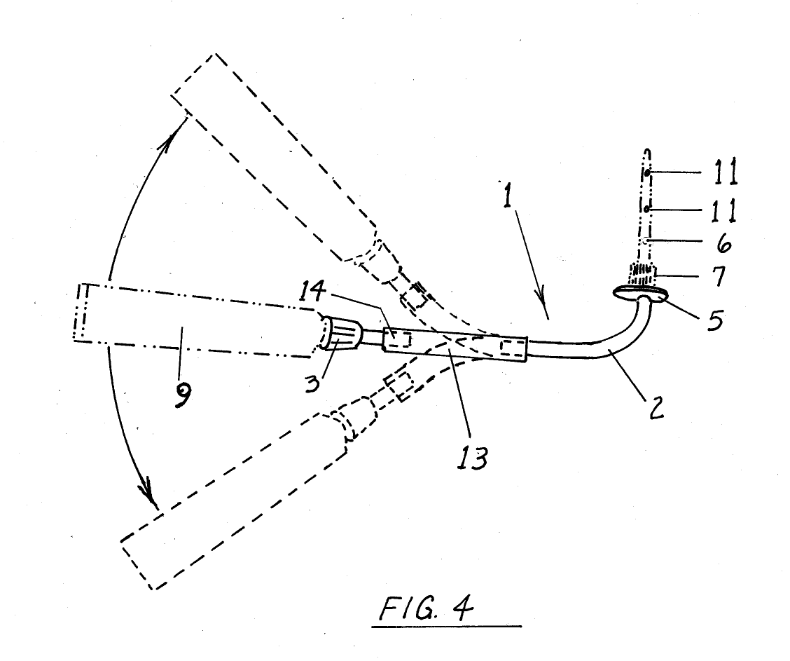

Although angles between 80 degrees and 100 degrees are suitable to hold the tube of ointment 9 in a substantially horizontal position while applying the ointment to the anal region for most users while seated on a toilet, some users may find other angles appropriate as well due to variations in seating, weight, body type, point of application (e.g., to the exterior of the anus, intrarectally, vaginally, etc.) or a desire to hold the tube above horizontal (see generally FIGS. 5 and 6). For example, FIG. 3 shows an embodiment of the applicator extension 1 wherein the angle is approximately 135 degrees. This configuration allows the user 12 to apply ointment while holding the tube of ointment 9 above the horizontal and allows the user 12 to keep their hands up and out of the bowl more easily (see FIG. 6). In another embodiment, shown in FIG. 4, at least a portion of the elongate tube 2 nearest said first end adjacent to the first end fitting 3 is made of a flexible tube. This flexible tube portion 13 could be permanently attached to the first end fitting 3 or, preferably, the first end fitting 3 would have a small protruding nib 14 and the flexible tube portion 13 would be removably attached to the nib 14 and to the elongate tube 2. The preferred method of attachment would be for the flexible tube portion 13 to be elastically slipped over the nib 14 (or the opening 10) and the first end of the elongate tube 2. Such embodiments would allow the user additional length and flexibility with regard to the positioning of the applicator 6. These additional lengths could be configured with adapted end fittings of their own or could simply be configured such that the ends of the additional lengths would elastically slip over the ends of the elongate tube 2 and the applicator 6 forming a sufficiently tight seal therewith. Thus, embodiments of the device may comprise nearly any angle desired by the user. In addition to the materials above, the elongate tube 2 or a portion thereof could comprise accordion-type pleats as is known in the art. The accordion-type pleats allow the elongate tube 2 to be bent in an amount desired by the user rather than a substantially fixed angle. In one such embodiment using material known in the state of the art, the elongate tube 2 may be constructed so as to snap into substantially rigid positions in multiple increments of approximately 15 degrees to a cumulative total angle of 135 degrees. Other materials allow for nearly infinite adjustability by the user to almost any desired angle. For example, the elongate tube 2 or a portion thereof could be manufactured of flexible non-rebound tubing or flexible stay-put tubing as is known in the art. Such tubing is constructed such that once the elongate tube 2 is bent into the desired position by the user, the properties of the tubing allow the elongate tube 2 to hold its position and shape. The non-rebound or stay-put tubing can be metallic, plastic or a combination thereof. For example, one such tubing is constructed of an aluminum inner material and a polyethylene outer material. Another comprises strip wound metallic tubing. Other flexible tubing with the desired non-rebound or stay-put properties known in the art may be used as well.

While I have shown and described certain embodiments of the present invention, it should be understood that the same is subject to many modifications and changes by those skilled in the art without departing from the basic concepts and operating principles of the disclosure.

What is claimed is:

1. An applicator extension for dispensing ointment from a tube of ointment through an applicator to the anal-genital area, said applicator extension comprising:
An elongate tube bent at an angle with a first end terminating in a first end fitting configured to be removably attached to the opening of said tube of ointment and a second end terminating in a second end fitting configured to be removably attached to said applicator.

2. The applicator extension of claim 1 wherein said angle is approximately 90 degrees.

3. The applicator extension of claim 1 wherein said first end fitting is a female threaded fitting configured to screw onto a male threaded fitting of said opening of said tube of ointment and said second end fitting is a male threaded fitting configured to screw into a female threaded fitting of said applicator.

4. The applicator extension of claim 1 wherein said first end fitting and said second end fittings are respectively releasably attached to said first end of said elongate tube and said second end of elongate tube.

5. The applicator extension of claim 1 further comprising:
a flange circumferentially fixed on the outer surface of said elongate tube adjacent to said second end.

6. The applicator extension of claim 1 wherein said angle is user adjustable.

7. The applicator extension of claim 6 wherein at least part of said elongate tube comprises non-rebound tubing.

8. The applicator extension of claim 6 wherein at least part of said elongate tube comprises stay-put tubing.

9. The applicator extension of claim 6 wherein a flexible tube is attached to said first end of said elongate tube.

10. The applicator extension of claim 6 wherein at least part of said elongate tube comprises accordion-type pleats allowing said part of said elongate tube to be snapped into varying angles.

11. The applicator extension of claim 6 wherein said first end fitting is a female threaded fitting configured to screw onto a male threaded fitting of said opening of said tube of ointment and said second end fitting is a male threaded fitting configured to screw into a female threaded fitting of said applicator.

12. The applicator extension of claim 6 further comprising:
a flange circumferentially fixed on the outer surface of said elongate tube adjacent to said second end.

13. The applicator extension of claim 6 wherein said first end fitting and said second end fittings are respectively releasably attached to said first end of said elongate tube and said second end of said elongate tube.

14. An applicator extension for dispensing ointment from a tube of ointment through an applicator to the anal genital area, said applicator extension comprising:
an adjustably bendable elongate tube having a first end fitting configured to be removably attached to the opening of said tube of ointment and a second end terminating in a second end fitting configured to be removably attached to said applicator.

15. The applicator extension of claim 14 wherein at least a portion of said elongate tube comprises non-rebound tubing.

16. The applicator extension of claim 14 wherein at least a portion of said elongate tube comprises stay-put tubing.

17. The applicator extension of claim 14 further comprising:
a flange circumferentially fixed on the outer surface of said elongate tube adjacent to said second end.

18. The applicator extension of 14 wherein said first end fitting and said second end fitting are respectively releasably attached to said first end of said elongate tube and said second end of said elongate tube.

19. The applicator extension of 14 wherein a flexible tube is attached to said first end of said elongate tube.
